(19) United States
(12) Patent Application Publication
(10) Pub. No.: US 2026/0053782 A1
(43) Pub. Date: Feb. 26, 2026

(54) TREATMENT REGIMENS FOR PARKINSON'S DISEASE

(71) Applicant: BIAL - PORTELA & CA, S.A., São Mamede Do Coronado (PT)

(72) Inventors: Patricio Soares Da Silva, São Mamede Do Coronado (PT); José Francisco Da Costa De Pinho Rocha, São Mamede Do Coronado (PT)

(21) Appl. No.: 19/104,241

(22) PCT Filed: Aug. 17, 2023
(86) PCT No.: PCT/PT2023/050025

§ 371 Date: Feb. 17, 2025

(30) Foreign Application Priority Data
Aug. 18, 2022 (GB) 2212082.8

(51) Int. Cl. A61K 31/4439; A61K 31/165; A61K 31/198; A61P 25/16

(52) U.S. Cl. CPC A61K 31/4439; A61K 31/165; A61K 31/198; A61P 25/16

(57) ABSTRACT

A method of treating Parkinson's disease in a patient who is receiving N doses of levodopa per day to provide a total daily dose of X mg of levodopa and who is starting to experience motor fluctuations or starting to show signs of “wearing-off”, the treatment comprising administering more than N doses of levodopa per day to provide a total daily dose of X mg of levodopa and administering a single daily dose of Y mg of opicapone, wherein X is from 100 to 1000, N is from 2 to 10 and Y is from 25 to 50.

### FIELD OF THE INVENTION

This invention relates to an optimized dosing regimen of levodopa and a COMT inhibitor for the treatment of Parkinson's disease in patients who are starting to experience end-of-dose motor fluctuations (or starting to show signs of “wearing-off”).

### BACKGROUND OF THE INVENTION

Levodopa (L-DOPA) has been used in clinical practice for several decades in the symptomatic treatment of various conditions, including Parkinson's disease. L-DOPA is able to cross the blood-brain barrier, where it is then converted to dopamine by the enzyme dopamine decarboxylase (DDC), thus increasing dopamine levels in the brain. However, conversion of L-DOPA to dopamine may also occur in peripheral tissues, possibly causing adverse effects. Therefore, it has become standard clinical practice to co-administer a peripheral DDC inhibitor (DDCI), such as carbidopa or benserazide, which prevents conversion to dopamine in peripheral tissues.

During early stages of Parkinson's disease, levodopa/DDCI therapy can almost entirely suppress symptoms of Parkinson's disease until the next dose is administered. However, most patients receiving long-term levodopa/DDCI will develop motor complications, such as end-of-dose motor fluctuations and dyskinesia, beyond the early stages of Parkinson's disease in spite of continued or increased levodopa administration (Aquino C C, Fox S H, Mov. Disord., 2015, 30, 80-89). This phenomenon is sometimes called “wearing off”. Patients often report spending several hours per day with end-of-dose motor fluctuations in the so called “off” state and this can have a substantial effect on their quality of life (Chapuis S, Ouchchane L, Metz O, Gerbaud L, Durif et al., Mov. Disord. 2005, 20, 224-30). The development of motor complications, such as end-of-dose motor fluctuations, defines the transition from the early stage of Parkinson's disease to a more advanced stage of the disease. As such, the control of motor complications eventually becomes a key clinical need for almost all patients (Poewe W, Neurology, 2009, 72, S65-73).

End-of-dose fluctuations are linked to the short half-life of oral levodopa (about 60-90 min). Adjustments of levodopa dose size and/or frequency are common approaches to managing response fluctuations. However, increasing the levodopa total daily dose by increasing dose frequency may worsen the severity of dyskinesia, while fragmenting the total daily dose into more frequent smaller doses may be associated with the intermittent re-emergence of symptoms due to oscillations of plasma concentration above and below the thresholds governing levodopa clinical response. As neither approach addresses the issue of the short half-life of conventional levodopa over the long term, an alternative pharmacological approach is to optimize levodopa delivery to the brain and manage levodopa-related complications by administering levodopa/DDCI with catechol-O-methyltransferase (COMT) inhibitors that increase the plasma elimination half-life of levodopa and decrease peak-to-trough variations.

Opicapone is a potent and long-acting COMT inhibitor that reduces the degradation of levodopa to the inactive metabolite 3-O-methyldopa. Opicapone is bioactive, bioavailable and exhibits low toxicity. Thus, opicapone has potentially valuable pharmaceutical properties in the treatment of some central and peripheral nervous system disorders where inhibition of COMT may be of therapeutic benefit, such as, for example, mood disorders; movement disorders, such as Parkinson's disease, parkinsonian disorders and restless legs syndrome; gastrointestinal disturbances; oedema formation states; and hypertension. The development of opicapone is described in L. E. Kiss et al, J. Med. Chem., 2010, 53, 3396-3411 and it was approved, in combination with L-DOPA, for the treatment of Parkinson's disease in the EU in June 2016, the US in April 2020 and Japan in June 2020 under the tradename “Ongentys”.

In the pivotal phase III clinical trials which supported these approvals, BIPARK-I (Ferreira et al., Lancet Neurol., 2016, 15, 154-65) and BIPARK-II (Lees et al., JAMA Neurol., 2017, 74, 197-206), the subjects' treatment regimens were not standardised prior to entering the trial. Accordingly, the subjects entered on a wide variety of levodopa regimens and the effect of the levodopa treatment regimen (both total daily dose and frequency of intakes) on the clinical effectiveness of opicapone in the management of motor fluctuations was not investigated.

A previously licensed COMT inhibitor, entacapone, was tested in patients suffering from early idiopathic Parkinson's disease, i.e. patients not suffering motor complications (Stocchi F et al. Initiating levodopa/carbidopa therapy with and without entacapone in early Parkinson disease: the STRIDE-PD study, Ann Neurol 2010: 68(1): 18-27). The STRIDE-PD trial is the only controlled trial to investigate the long-term optimized delivery of levodopa including the use of enzyme inhibitors for the prevention or delay of motor complications at the beginning of treatment in levodopa-naïve patients. The trial failed to demonstrate that initiating levodopa therapy with a COMT inhibitor (entacapone) can delay the time of onset or reduce the frequency of dyskinesia compared to levodopa-only therapy. In fact, the addition of entacapone was associated with a shorter time to onset of motor complications and increased frequency of dyskinesia.

Therefore, there remains a need for improvements in the safety and efficacy of treatment of Parkinson's disease when a patient's symptoms are no longer controlled by levodopa, without inducing motor complications from excessive dosing with levodopa. In particular, there remains a need for a more effective regimen to treat Parkinson's disease in a patient who is starting to experience end-of-dose motor fluctuations (or starting to show signs of “wearing-off”).

The present invention arose from the results of a clinical study undertaken by the inventors. Details of the trial design were published as an abstract in the European Journal of Neurology in 2021 (J. Ferreira et al, Eur. J. Neurol. 2021, 28 (Suppl. 1), 558-752), and were presented virtually at the 7^{th} Congress of the European Academy of Neurology (EAN) on 19-22 Jun. 2021. No results were presented.

The pharmacokinetic data were published as an abstract in the European Journal of Neurology in June 2022 (J. Ferreira et al, Eur. J. Neurol. 2022, 29 (Suppl. 1), 440-441), and were presented virtually at the 8^{th} Congress of the European Academy of Neurology (EAN) on 25-27 Jun. 2022. No clinical results were presented.

The clinical data were published as a journal article in the journal Movement Disorders on 31 Aug. 2022 (J. Ferreira et al, Mov. Disord. 2022, 37, 2272-2283).

### SUMMARY OF THE INVENTION

The present inventors have discovered an optimized dosing regimen of levodopa and a COMT inhibitor for the treatment of Parkinson's disease, in particular idiopathic Parkinson's disease, in patients who are starting to experience end-of-dose motor fluctuations (or starting to show signs of “wearing-off”).

Accordingly, in a first general embodiment, the invention provides a method of treating Parkinson's disease in a patient who is receiving N doses of levodopa per day to provide a total daily dose of X mg of levodopa and who is starting to experience end-of-dose motor fluctuations or starting to show signs of “wearing-off”, the treatment comprising administering more than N doses of levodopa per day to provide a total daily dose of X mg of levodopa, and administering a single daily dose of Y mg of opicapone; wherein: X is from 100 to 1000, N is from 2 to 10 and Y is from 25 to 50.

In a second general embodiment, the invention provides opicapone and levodopa for use in the treatment of Parkinson's disease in a patient who is receiving N doses of levodopa per day to provide a total daily dose of X mg of levodopa and who is starting to experience motor fluctuations or starting to show signs of “wearing-off”, the treatment comprising administering more than N doses of levodopa per day to provide a total daily dose of X mg of levodopa and administering a single daily dose of Y mg of opicapone, wherein X is from 100 to 1000, N is from 2 to 10 and Y is from 25 to 50.

In a third general embodiment, the invention provides the use of opicapone and levodopa in the manufacture of medicaments for the treatment of Parkinson's disease in a patient who is receiving N doses of levodopa per day to provide a total daily dose of X mg of levodopa and who is starting to experience motor fluctuations or starting to show signs of “wearing-off”, the treatment comprising administering more than N doses of levodopa per day to provide a total daily dose of X mg of levodopa and administering a single daily dose of Y mg of opicapone, wherein X is from 100 to 1000, N is from 2 to 10 and Y is from 25 to 50.

### BRIEF DESCRIPTION OF THE DRAWINGS

The invention will now be described in detail with reference to the accompanying drawings, in which:

FIG. 1 shows the design of a clinical study. LD=levodopa; CD=carbidopa; PK=pharmacokinetics; R=randomisation: V1-V4=Visit 1-Visit 4.

FIG. 2 shows the mean levodopa plasma profile versus time following a 2-week 5-intake (every 3 hours) daily oral administration of LD/CD 500/125 mg compared with a 2-week 4-intake (every 4 hours) daily oral administration of LD/CD 400/100 mg plus 50 mg opicapone (A) or compared with a 2-week 5-intake (every 3 hours) daily oral administration of LD/CD 400/100 mg plus 50 mg opicapone (B); LD/CD=levodopa/carbidopa.

FIG. 3 shows the 12-hour ON-/OFF-time data reported on pharmacokinetics days superimposed to the mean levodopa plasma profile versus time following: 2-week 5-intake (every 3 hours) daily oral administration of levodopa/carbidopa 500/125 mg without opicapone (A) compared with 2-week 4-inake (every 4 hours) daily oral administration of levodopa/carbidopa 400/100 mg plus once-daily opicapone, 50 mg (B); and 2-week 5-intake (every 3 hours) daily oral administration of levodopa/carbidopa 500/125 mg without opicapone (C) compared with 2-week 5-intake (every 3 hours) daily oral administrations of levodopa/carbidopa 400/100 mg plus once-daily opicapone, 50 mg (D); LD/CD=levodopa/carbidopa; OPC=opicapone; arrows=time to ON; bars=ON state periods; solid vertical line=time of Best ON.

FIG. 4 is a schematic diagram which shows typical levodopa pharmacokinetics; Cₘₐₓ=highest (rise, peak) levodopa concentration level found in plasma; Cₘᵢₙ=lowest (fall, trough) levodopa concentration level found in plasma; Fluctuation Index (FI)=magnitude of the rise and fall of levodopa level in plasma calculated as [(Cₘₐₓ−Cₘᵢₙ)/C_{avg}]*100; AUC (area under the curve)=systemic exposure of levodopa in plasma as a function of time.

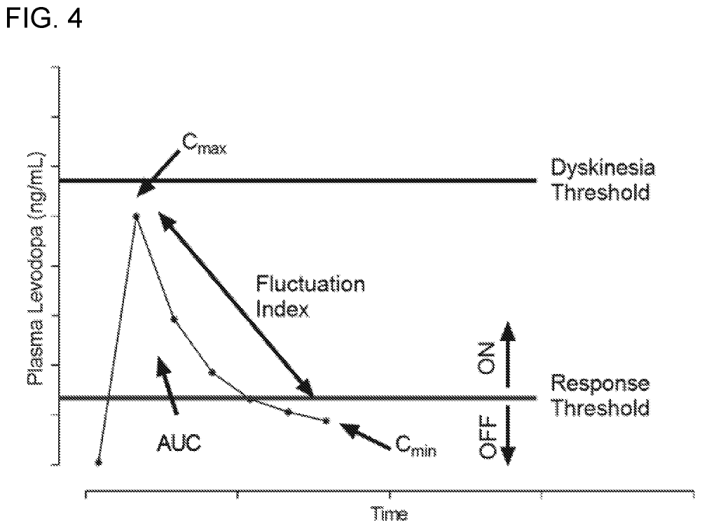

### DETAILED DESCRIPTION OF THE INVENTION

### A. Definitions

The following definitions apply to the terms as used throughout this specification unless otherwise limited in specific instances.

The term “idiopathic Parkinson's disease” encompasses most (80-85%) Parkinson's disease (diagnosed according to either the United Kingdom Parkinson's Disease Society Brain Bank Clinical Diagnostic Criteria or the Movement Disorder Society criteria) and excludes atypical parkinsonism, secondary [acquired or symptomatic] parkinsonism and Parkinson-plus syndrome, for example, drug-induced parkinsonism, vascular parkinsonism, normal pressure hydrocephalus, corticobasal degeneration, progressive supranuclear palsy and multiple system atrophy. It typically involves prominent bradykinesia and variable associated extrapyramidal signs and symptoms. It is typically accompanied by degeneration of the nigrostriatal dopaminergic system, with neuronal loss and reactive gliosis in the substantia nigra found at autopsy. In idiopathic Parkinson's disease, α-synuclein typically accumulates in neuronal perikarya (Lewy bodies) and neuronal processes (Lewy neurites).

The term “GBA1-carrying Parkinson's disease” or “GBA-associated parkinsonism” relates to a specific group of Parkinson's disease patients with a mutation in both copies of the GBA1 gene that codes for the Glucocerebrosidase (GCase) enzyme. These heterozygous mutations are associated with a higher risk for developing Parkinson's disease. Approximately 10% of the overall patient population in the U.S. with clinically diagnosed Parkinson's disease carries a GBA1 mutation.

The term “symptoms of Parkinson's disease” includes both motor symptoms (e.g. tremor, rigidity, bradykinesia and postural instability) and non-motor symptoms (e.g. cognitive changes, gastrointestinal symptoms, loss of sight, taste and/or smell, pain, fatigue, light-headedness, sexual problems, sleep disorders and weight loss). Such symptoms can be assessed using one or more of the symptomatic readouts described below including clinical assessments (e.g., 12-hour and 24-hour diaries, and PGI-C).

The term “motor complications” relates to Parkinson's disease motor symptoms which are related to levodopa therapy. They arise when levodopa/DDCI therapy alone no longer provides complete control of the patient's symptoms. They include motor fluctuations and/or dyskinesia. Motor complications are sustained, but not necessarily regular or predictable, such that they quantifiably and negatively impact on the patient's quality of life (QoL). “Clinically diagnosed motor complications” generally result in a total score of MDS-UPDRS Part IV A+B+C greater than 6, preferably greater than 3, more preferably greater than 0 and/or one or more positive symptoms in the 9-items Wearing off Questionnaire (WOQ-9). A total score of MDS-UPDRS Part IV A+B+C greater than 0 (zero) is the most preferred definition of clinically diagnosed motor complications. It should be noted that motor complications can be the same as motor symptoms of Parkinson's disease. However, a motor symptom which is initially treatable by levodopa/DDCI therapy, but which re-emerges at a later stage of disease in spite of maintaining levodopa/DDCI therapy, is then considered a motor complication.

The term “motor fluctuations” includes end-of-dose fluctuations (also known as the wearing-off phenomenon), paradoxical fluctuations and unpredictable ON/OFF periods.

The term “OFF period” or “OFF episode” is defined as the times during which a patient treated with levodopa no longer experiences its symptomatic benefit and is said to be in an “OFF” state. On the other hand, the term “ON period” or “ON episode” is defined as the times when a patient treated with levodopa experiences its symptomatic benefit the patient is said to be in an “on” state.

The term “time to best ON” is the time until the patient experiences a maximum symptomatic effect after administration of a dose of levodopa/DDCI therapy.

The term “end-of-dose motor fluctuations”, also known as the “wearing off” phenomenon, relates to the predictable re-emergence or worsening of symptoms before administration of the next dose of levodopa/DDCI therapy. Typically, such re-emergence or worsening of symptoms starts 3-4 hours after a dose of levodopa, as the medication wears off and symptoms re-emerge or worsen. Symptoms then typically improve 15-45 minutes after the next levodopa dose is taken.

A patient “starting to experience motor fluctuations” or “starting to show signs of “wearing-off”” includes a patient with a clinically recognised duration of motor fluctuations of up to 3 years, including up to 2 years, or even up to 1 year. Patients with a duration of motor fluctuations of up to 1 year are known as “early motor fluctuators”. Typically, a patient is “starting to experience motor fluctuations” when the patient is treated with levodopa and starts to experience detectable “OFF periods” or “OFF episodes”.

The term “dyskinesia” or “levodopa-induced dyskinesia” includes peak dose dyskinesia, diphasic dyskinesia and OFF dyskinesia. Common symptoms include chorea and dystonia. Less common symptoms include akathasia (excessive motor restlessness), a high stepped overshooting gait, rapid alternating movements (RAM) of legs, blepharospasm, and mixed pattern of abnormal movements (Fahn S., Ann. Neurol., 2000, 47, S2-S9).

The term ““ON” with troublesome dyskinesia” is a period after administration of a dose of levodopa/DDCI therapy when the patient has limited mobility due to the presence of dyskinesia, but experiences some symptomatic relief consistent with an “ON period”.

The term “adjunctive therapy”, also known as “adjunct therapy”, “add-on therapy”, or “adjuvant care”, is therapy that is given in addition to the primary or initial therapy to maximize its effectiveness. In the current application, levodopa is the primary therapy and the DCCI and COMT inhibitor (i.e. opicapone) are the adjunctive therapies.

The term “treatment-emergent adverse event” is defined as any event not present before exposure to the study drug or any event already present that worsens in either intensity or frequency after first intake of study drug until 2 weeks after last intake of the study drug.

The term “maximum observed plasma concentration” or “Cₘₐₓ” is the highest (rise, peak) drug concentration level found in plasma. In the current application, it is the highest levodopa concentration level found in plasma. An increase in Cm results in an increase in the risk of developing dopamine induced side effects, such as dyskinesia.

The term “minimum observed plasma concentration” or “Cₘᵢₙ” is the lowest (fall, trough) drug concentration level found in plasma. In the current application, it is the lowest levodopa concentration level found in plasma. An increase in Cₘᵢₙ results in a decrease in the time taken to achieve or maintain ON state. If the increase observed for Cₘₐₓ is not in the same or higher proportions as the increase observed in Cₘᵢₙ, then the risk of developing motor complications is decreased.

The term “average observed plasma concentration” or “C_{avg}” is the average plasma drug concentration during a dosage interval at steady-state.

The term “fluctuation index” or “FI” is the magnitude of the rise and fall of drug level in the plasma, calculated as [(Cₘₐₓ−Cₘᵢₙ)/C_{avg}]*100. In the current application, it is rise and fall of levodopa level in plasma. An increase in FI results in an increase in the risk of developing motor complications.

The term “area under the curve” or “AUC” is systemic exposure of drug in plasma as a function of time. In the current application, it is the systemic exposure of levodopa in plasma as a function of time. An increase in AUC results in an increase in the time spent in an ON state (i.e. a decrease in the time spent in an OFF state).

Other variations to the disclosed embodiments can be understood and effected by those skilled in the art in practicing the claimed invention, from a study of the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

### B. Treatment of Parkinson's Disease in a Patient Starting to Experience End-of-Dose Motor Fluctuations (or Starting to Show Signs of “Wearing Off”)

The present invention provides a dosing regimen of levodopa plus opicapone in the treatment of Parkinson's disease, in particular idiopathic Parkinson's disease, in patients starting to experience end-of-dose motor fluctuations (or starting to show signs of “wearing off”).

In a first general embodiment, the invention provides a method of treating Parkinson's disease in a patient who is receiving N doses of levodopa per day to provide a total daily dose of X mg of levodopa and who is starting to experience end-of-dose motor fluctuations or starting to show signs of “wearing-off”, the treatment comprising administering more than N doses of levodopa per day to provide a total daily dose of X mg of levodopa, and administering a single daily dose of Y mg of opicapone; wherein: X is from 100 to 1000, N is from 2 to 10 and Y is from 25 to 50.

In a second general embodiment, the invention provides opicapone and levodopa for use in the treatment of Parkinson's disease in a patient who is receiving N doses of levodopa per day to provide a total daily dose of X mg of levodopa and who is starting to experience motor fluctuations or starting to show signs of “wearing-off”, the treatment comprising administering more than N doses of levodopa per day to provide a total daily dose of X mg of levodopa and administering a single daily dose of Y mg of opicapone, wherein X is from 100 to 1000, N is from 2 to 10 and Y is from 25 to 50.

In a third general embodiment, the invention provides the use of opicapone and levodopa in the manufacture of medicaments for the treatment of Parkinson's disease in a patient who is receiving N doses of levodopa per day to provide a total daily dose of X mg of levodopa and who is starting to experience motor fluctuations or starting to show signs of “wearing-off”, the treatment comprising administering more than N doses of levodopa per day to provide a total daily dose of X mg of levodopa and administering a single daily dose of Y mg of opicapone, wherein X is from 100 to 1000, N is from 2 to 10 and Y is from 25 to 50.

The following embodiments are compatible with the general embodiments and with each other:

In another embodiment, the Parkinson's disease is idiopathic Parkinson's disease or GBA1-carrying Parkinson's disease. In a preferred embodiment, the Parkinson's disease is idiopathic Parkinson's disease, which accounts for the majority of Parkinson's disease.

In another embodiment, N is 3 to 6, preferably N is 3 to 5, more preferably 3 to 4. In an even more preferred embodiment, N is 4.

In another embodiment, “more than N doses” is N+1 or N+2 doses. In a preferred embodiment, “more than N doses” is N+1 doses.

In another embodiment, X is 300 to 600, preferably 300 to 500, more preferably 300 to 400. In an even more preferred embodiment, X is 400.

In another embodiment, Y is 25 or 50. For example, Y is 25. For example, Y is 50.

In a specific preferred embodiment, the patient is receiving 4 doses of levodopa to provide a total daily dose of 400 mg per day and the treatment comprises administering 5 doses of levodopa per day to provide a total daily dose of 400 mg of levodopa and administering a single daily dose of 50 mg of opicapone.

In another embodiment, the levodopa dosage amount is not the same for every dose. For example, the levodopa dosage amount is higher for odd numbered doses. For example, the levodopa dosage amount is higher for even numbered doses. Standard levodopa dosage amounts include 250 mg, 100 mg, 75 mg or 50 mg. Most patients take a single amount of levodopa for every dose, for example, 100 mg for every dose.

In a specific more preferred embodiment, the patient is receiving 4 doses of levodopa to provide a total daily dose of 400 mg per day and the treatment comprises administering 5 doses of levodopa per day to provide a total daily dose of 400 mg of levodopa and administering a single daily dose of 50 mg of opicapone, wherein the 5 doses of levodopa to provide a total daily dose of 400 mg of levodopa are administered in the following dosing regimen: 100 mg, 50 mg, 100 mg, 50 mg, and 100 mg.

In another embodiment, the single daily dose of opicapone is administered at least 1 hour after the last dose of levodopa.

In another embodiment, the patient is receiving N doses of a dopamine decarboxylase inhibitor (DDCI) to provide a total daily dose of Z mg of the DDCI, the method further comprising administering more than N doses of the DDCI per day to provide a total daily dose of Z mg of DDCI, wherein Z is from 50 to 250, preferably 75 to 150.

In another embodiment, the DDCI is carbidopa or benserazide. For example, the DDCI is carbidopa. For example, the DDCI is benserazide.

In another embodiment, the DDCI doses are administered together with the levodopa doses.

In a specific preferred embodiment, the patient is receiving 4 doses of carbidopa to provide a total daily dose of 100 mg per day and the treatment comprises administering 5 doses of carbidopa per day to provide a total daily dose of 100 mg of carbidopa.

In a specific preferred embodiment, the patient is receiving 4 doses of levodopa/carbidopa to provide a total daily dose of 400/100 mg of levodopa/carbidopa per day and the treatment comprises administering 5 doses of levodopa/carbidopa per day to provide a total daily dose of 400/100 mg of levodopa/carbidopa and administering a single daily dose of 50 mg of opicapone.

In a specific more preferred embodiment, the patient is receiving 4 doses of levodopa/carbidopa to provide a total daily dose of 400/100 mg per day and the treatment comprises administering 5 doses of levodopa/carbidopa per day to provide a total daily dose of 400/100 mg of levodopa/carbidopa and administering a single daily dose of 50 mg of opicapone, wherein the 5 doses of levodopa/carbidopa to provide a total daily dose of 400/100 mg of levodopa/carbidopa are administered in the following dosing regimen: 100/25 mg, 50/12.5 mg, 100/25 mg, 50/12.5 mg, and 100/25 mg.

In another embodiment, the patient starting to experience motor fluctuations or starting to show signs of “wearing-off” is a patient with a duration of motor fluctuations of up to 3 years, preferably up to 2 years, more preferably up to 1 year.

The results described below confirm that the addition of opicapone and splitting one or more levodopa dosage results in an improved treatment for Parkinson's disease (e.g., idiopathic Parkinson's disease) in patients who are otherwise not receiving full therapeutic benefit. The discovery is encompassed by the three general embodiments described above. Based on these data, it is anticipated that patients could reduce the total dosage of levodopa (and DCCI) by the addition of opicapone and splitting one or more levodopa dosage.

For example, it is anticipated the invention could provide a method of treating Parkinson's disease in a patient who is receiving N doses of levodopa per day to provide a total daily dose of X mg of levodopa and who is starting to experience end-of-dose motor fluctuations or starting to show signs of “wearing-off”, the treatment comprising administering a total daily dose of less than X mg of levodopa per day, and administering a single daily dose of Y mg of opicapone; wherein: X is from 100 to 1000, N is from 2 to 10, Y is from 25 to 50 and “less than X mg of levodopa per day” involves a reduction of at least X/2N mg, preferably “less than X mg of levodopa per day” involves a reduction of at least X/N mg; the method excludes treating Parkinson's disease in a patient who is starting to experience end-of-dose motor fluctuations or starting to show signs of “wearing-off” and who is receiving 5 doses of levodopa to provide a total daily dose of 500 mg per day, the treatment comprising administering 5 doses of levodopa per day to provide a total daily dose of 400 mg of levodopa and administering a single daily dose of 50 mg of opicapone, wherein the 5 doses of levodopa provide a total daily dose of 400 mg of levodopa and are administered in the following dosing regimen: 100 mg, 50 mg, 100 mg, 50 mg, and 100 mg. Preferably, the method involves more than one individual dosage amount (e.g., two individual dosage amounts, such as a combination of 50 mg and 100 mg levodopa during the day).

Specific examples supported by the results include:

A method of treating Parkinson's disease in a patient who is starting to experience end-of-dose motor fluctuations or starting to show signs of “wearing-off” and who is receiving 3 doses of levodopa to provide a total daily dose of 300 mg per day, the treatment comprising administering 3 doses of levodopa per day to provide a total daily dose of 200 mg of levodopa and administering a single daily dose of 50 mg of opicapone, wherein the 3 doses of levodopa provide a total daily dose of 200 mg of levodopa and are administered in the following dosing regimen: 100 mg, 50 mg, and 50 mg.

A method of treating Parkinson's disease in a patient who is starting to experience end-of-dose motor fluctuations or starting to show signs of “wearing-off” and who is receiving 4 doses of levodopa to provide a total daily dose of 400 mg per day, the treatment comprising administering 4 doses of levodopa per day to provide a total daily dose of 300 mg of levodopa and administering a single daily dose of 50 mg of opicapone, wherein the 4 doses of levodopa provide a total daily dose of 300 mg of levodopa and are administered in the following dosing regimen: 100 mg, 50 mg, 100 mg, and 50 mg.

A method of treating Parkinson's disease in a patient who is starting to experience end-of-dose motor fluctuations or starting to show signs of “wearing-off” and who is receiving 3 doses of levodopa to provide a total daily dose of 300 mg per day, the treatment comprising administering 4 doses of levodopa per day to provide a total daily dose of 250 mg of levodopa and administering a single daily dose of 50 mg of opicapone, wherein the 4 doses of levodopa provide a total daily dose of 250 mg of levodopa and are administered in the following dosing regimen: 100 mg, 50 mg, 50 mg, and 50 mg.

A method of treating Parkinson's disease in a patient who is starting to experience end-of-dose motor fluctuations or starting to show signs of “wearing-off” and who is receiving 4 doses of levodopa to provide a total daily dose of 400 mg per day, the treatment comprising administering 5 doses of levodopa per day to provide a total daily dose of 300 mg of levodopa and administering a single daily dose of 50 mg of opicapone, wherein the 5 doses of levodopa provide a total daily dose of 300 mg of levodopa and are administered in the following dosing regimen: 100 mg, 50 mg, 50 mg, 50 mg, and 50 mg.

### C. Clinical Protocol

The invention was discovered through a Phase IL, randomized, open-label trial evaluating the effect of opicapone (50 mg once-daily) on L-DOPA pharmacokinetics in Parkinson's disease patients with end-of-dose motor fluctuations taking different levodopa/carbidopa (LD/CD) sparing regimens (EudraCT number: 2020-003139-12).

Subjects were aged ≥30 years with a clinical diagnosis of idiopathic Parkinson's disease according to the United Kingdom Parkinson's Disease Society Brain Bank Clinical Diagnostic Criteria with disease severity stage 1-3 (according to modified Hoehn & Yahr staging) at ON and with end-of-dose motor fluctuations and signs of “wearing off”. Subjects were receiving treatment with levodopa/DDCI for at least 1 year with clear clinical improvement.

The Hoehn and Yahr scale is used to describe the progression of Parkinson disease symptoms. The original version (Hoehn M., Yahr M., Neurology, 1967, 17, 427-42) included stages 1 to 5. The modified version includes additional stages 1.5 and 2.5 to allow recording of the intermediate stages of Parkinson's disease.

Although there were no specific requirements in terms of levodopa dose frequency, all patients recruited were already receiving a daily treatment regimen of 5-intake LD/CD 500/125 mg. Patients were excluded if they were experiencing severe and/or unpredictable ‘OFF’ periods; had received treatment with sustained release levodopa/DDCI within the last four weeks prior to screening, were taking prohibited medication (neuroleptics, venlafaxine, monoamine oxidase (MAO) inhibitors [except selegiline, safinamide or rasagiline], or antiemetics with antidopaminergic action [except domperidone] or if they had previously received any COMT inhibitor (entacapone, tolcapone or opicapone).

### Identity of Investigational Products

Opicapone can be synthesised as described in WO 2013/089573 and formulated into 50 mg capsules as described in WO 2010/114405. Opicapone was taken orally once daily in the evening at least 1 hour after the last daily dose of L-DOPA/DDCI (considered the bedtime dose).

TABLE 1A
Opicapone (Ongentys)

| | |
|---|---|
| Active substance: | opicapone |
| Strength: | 50 mg hard capsules for oral administration |
| Manufacturer: | BIAL - Portela & C^{a}, S.A. |

TABLE 1B
Levodopa/Carbidopa (fixed combination)

| | |
|---|---|
| Active substance: | L-3,4-dihydroxyphenylalanine (L-DOPA)/carbidopa |
| Strength: | 100 mg/25 mg and 50 mg/12.5 mg tablets for oral |
| | administration |
| Manufacturer: | Central Pharmacy at PRA Health Sciences, |
| | Netherlands |

### Clinical Trial Design

Details of the trial design, as set out below, were published as an abstract in the European Journal of Neurology in 2021 (J. Ferreira et al, Eur. J. Neurol. 2021, 28 (Suppl. 1), 558-752), and was presented virtually at the 7^{th} Congress of the European Academy of Neurology (EAN) on 19-22 Jun. 2021. No results were presented.

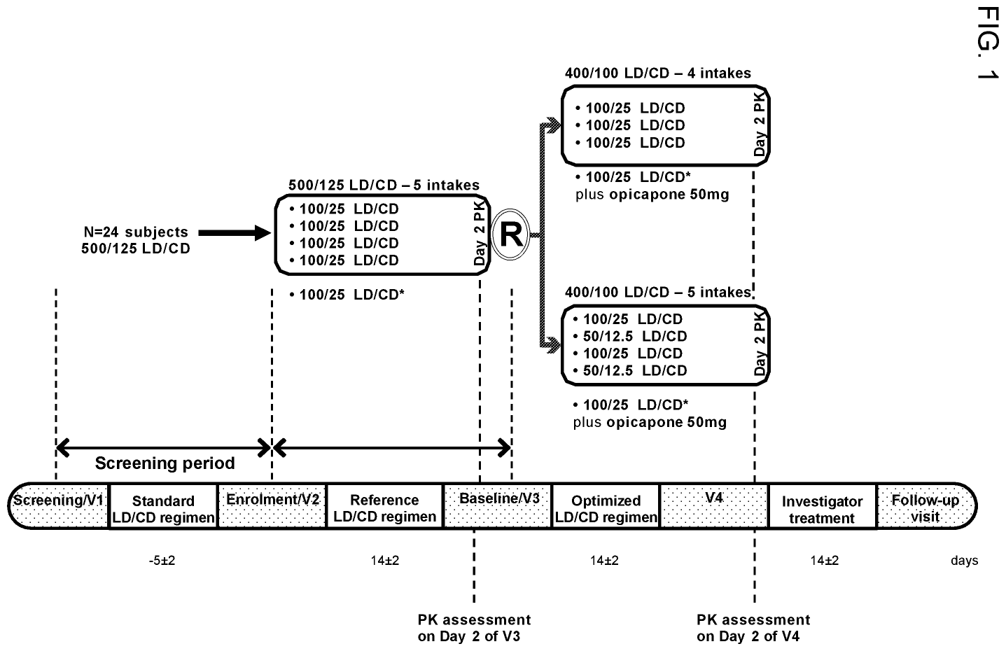

The study design is illustrated in FIG. 1.

The eligible subjects received the following study treatments during the different study periods:

### Period 1

Following screening, all subjects received an LD/CD dose and regimen of 100/25 mg QD5 (giving a total daily dose of 500/125 mg) fixed every 3 hours (from 8:00 to 20:00) for 14±2 days. This is the reference regimen.

### Period 2

Patients were then randomized (1:1) to receive one of two different treatment regimens of opicapone 50 mg plus LD/CD (400/100 mg daily) for an additional 14±2 days.
Opicapone regimen #1: the subjects received a LD/CD dose and regimen of 100/25 mg QD4 (fixed every 4 hours from 8:00 to 20:00) with a total daily LD/CD dose of 400/100 mg plus 50 mg OPC QD at least 1 hour after the last administration of LD/CD.
Opicapone regimen #2: the subjects received an alternating LD/CD dose and regimen of 100/25 mg and 50/12.5 mg doses QD5 (fixed every 3 hours from 8:00 to 20:00) with a total daily LD/CD dose of 400/100 mg (i.e. 100/25 mg, 50/12.5 mg, 100/25 mg, 50/12.5 mg and 100/25 mg) plus 50 mg OPC QD at least 1 hour after the last administration of LD/CD.

### Period 3

After completing one of the two 2-week LD/CD 400/100 mg plus 50 mg opicapone regimens, the subjects were followed up for 1 to 2 weeks (post-study visits, POV).

### Pharmacokinetic Parameters

The primary endpoint was pharmacokinetic-based, and was to assess the effect of opicapone once daily on levodopa pharmacokinetics. The pharmacokinetic study endpoint was set after 2 weeks of each treatment regimen (i.e. at the end of the period 1 and at the end of period 2). Pharmacokinetic evaluation occurred over 12 hours with sampling every 30 minutes, except the last sample following each levodopa dose in opicapone regimen #1,which was sampled at 1 hour.

The pharmacokinetic parameters assessed for each of the regimens included maximum observed plasma concentration (Cₘₐₓ), time to achieve peak plasma levels (tₘₐₓ), minimum observed plasma concentration (Cₘᵢₙ), plasma elimination half-life (t_{1/2}), area under the curve (AUC) and fluctuation index (FI, as an expression of peak-to-trough fluctuation, Cₘₐₓ and Cₘᵢₙ relative to average concentration C_{avg}).

Pharmacokinetics of the levodopa metabolite, 3-O-methyldopa (3-OMD) was also evaluated.

The maximum Cₘₐₓ was observed over the first 3 or 4 LD/CD doses, the minimum Cₘᵢₙ was observed over the first 3 or 4 LD/CD doses, excluding the first pre-dose data, and the FI was also summarized for each treatment arm based on all patients with estimateable data (subjects must have had a minimum of 3 data points after Cₘₐₓ to estimate the terminal elimination rate constant required to estimate the t_{1/2}, C_{avg}, AUC and FI %).

### Clinical Assessments

Clinical and safety/tolerability outcomes were also assessed as exploratory secondary outcomes. Clinical outcomes included ON and OFF-time assessed using patient-rated tools. The timing of ON and OFF state was registered (in real time) by the investigators during the matching 12-hour pharmacokinetic evaluation, as such applied on the pharmacokinetics days only), and patients also completed 24-hour Hauser ON/OFF diary charts (in 30 minute blocks) during the three days before each pharmacokinetic visit. Diary states of interest were OFF-time and ON-time, including ON without dyskinesia, ON with non-troublesome dyskinesia and ON with troublesome dyskinesia. In addition, the patient global impression of change (PGI-C) of opicapone 50 mg was evaluated at the end of the LD/CD 400/100 mg plus opicapone treatment, compared to before the start of the opicapone treatment. Safety and tolerability endpoints included treatment-emergent adverse events (TEAEs), serious TEAEs and TEAEs leading to discontinuation.

All study assessments were evaluated for each LD/CD 400/100 mg plus 50 mg opicapone treatment regimen, compared to the LD/CD 500/125 mg without opicapone regimen.

### Patient ON/OFF Diary

The 24-hour patient Hauser ON/OFF diary charts is divided into 30-minute sections running from 00:00 to 23:30. During each 30 minute interval patients are asked to mark which of the states they are experiencing, namely, “ON without dyskinesia”, “ON with troublesome dyskinesia”, “ON with non-troublesome dyskinesia”, “OFF” and “ASLEEP”.

### Patient Global Impression of Change (PGI-C)

The Patient Global Impression of Change (PGI-C) is a 7-point scale that measures the change in a person's global impression relative to a baseline state at the beginning of the study. The following ratings are used to determine the change in the subjects global impression: “very much improved”; “much improved”; “minimally improved”; “no change”; “minimally worse”; “much worse”; or “very much worse”. People with improvement are those rated as “very much improved”, “much improved” or “minimally improved”.

For an individual subject, the PGI-C scale is preferably scored by the same investigator/rater throughout the study.

### D. Results and Discussion of Clinical Trial

### Overall Summary

Despite a reduced daily LD/CD dose (400/100 mg instead of 500/125 mg), the addition of 50 mg opicapone at least doubled the plasma half-life (tin) of levodopa when compared to LD/CD 500/125 mg without opicapone regimen. The impact on t_{1/2} triggered two immediate consequences: a similar significant 2-fold increase in levodopa trough concentrations (Cₘᵢₙ) and a corresponding significant ˜30% increase in levodopa systemic exposure (AUCₜₒₜₐₗ).

Adding opicapone had no impact on the time to reach (tₘₐₓ) maximum levodopa concentrations (Cₘₐₓ). Although no significant impact on Cₘₐₓ was observed when adding 50 mg opicapone to either of the opicapone regimen #1 or #2, a non-significantly higher (15%) levodopa Cₘₐₓ was observed with the 4-intake LD/CD 400/100 mg plus 50 mg opicapone regimen (opicapone regimen #1).

The increase in levodopa Cₘᵢₙ combined with a steady Cₘₐₓ resulted in reduced (up to ˜40%) levodopa plasma fluctuations, as assessed by the levodopa fluctuation index (FI). The 5-intake LD/CD 400/100 mg plus 50 mg opicapone (opicapone regimen #2) achieved a statistically significant result.

The results suggest that regimens with shorter levodopa dosing intervals provide the smoothest levodopa pharmacokinetic profile with less fluctuation in the plasma levels of levodopa when compared with longer dosing intervals. Thus, increasing levodopa dosing frequency when adding opicapone as adjunct therapy may offer advantages in terms of control of motor fluctuations.

Modifying the pharmacokinetic profile of levodopa by the addition of opicapone was also associated with reduced OFF time and increased ON time.

PK Results for LD/CD 500/125 mg QD5 without Opicapone Compared with LD/CD 400/100 Mg QD4 Plus 50 mg Opicapone

The 4-intake LD/CD 400/100 mg plus 50 mg opicapone regimen provided a 15% increase in levodopa Cₘₐₓ (maximum Cₘₐₓ observed), which was not statistically significant; a statistically significantly increase in levodopa Cₘᵢₙ (˜2 fold, p=0.0016); and a non-significant difference in levodopa tₘₐₓ, compared to the 5-intake LD/CD 500/125 mg without opicapone regimen. There was also a 2-fold higher levodopa t_{1/2}, resulting in a corresponding statistically significant 27% increase in levodopa AUCₜₒₜₐₗ (p=0.0003). Although the levodopa FI was reduced compared to the 5-intake LD/CD 500/125 mg without opicapone regimen, the reduction of 10% was not statistically significant. See Tables 2 and 3 for results.

PK Results for LD/CD 500/125 mg QD5 without Opicapone Compared with LD/CD 400/100 Mg QD5 Plus 50 mg Opicapone

The 5-intake LD/CD 400/100 mg plus 50 mg opicapone regimen provided a statistically significantly higher levodopa Cₘᵢₙ(˜2.5 fold, p<0.0001); and a more than 2-fold longer levodopa t_{1/2}, resulting in a corresponding 29% increase in levodopa AUCₜₒₜₐₗ, which was statistically significant (p<0.0001). There were no significant differences in Cₘₐₓ or tₘₐₓ. The stabilized Cₘₐₓ together with the significantly increased Cₘᵢₙ led to a statistically significant, 40% lower levodopa FI ratio (last/evening levodopa/carbidopa intake was not included in analysis, p<0.0001). See Tables 2 and 3 for results.

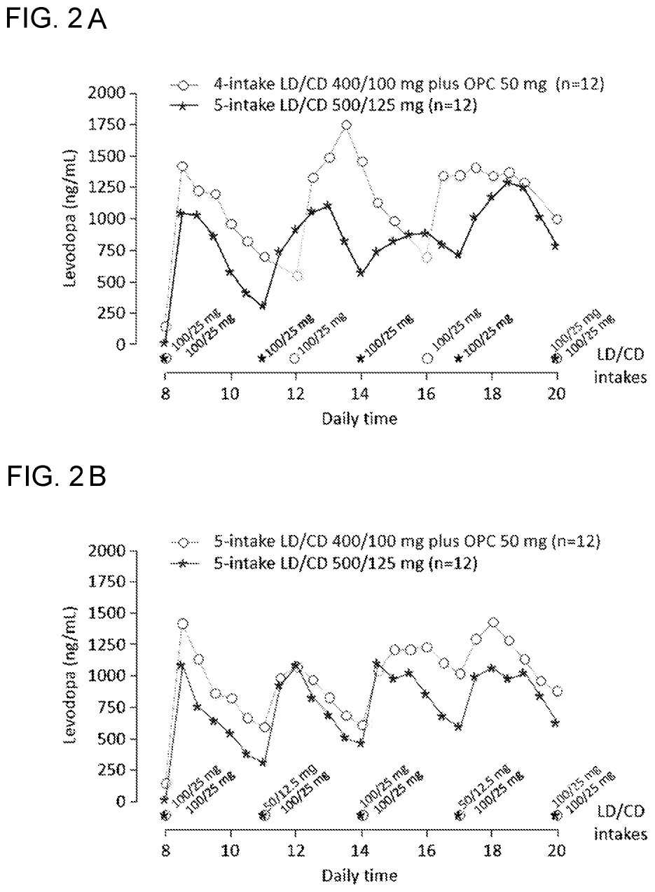

The results in FIG. 2 demonstrate that regimens with the shorter levodopa dosing interval presented the smoothest levodopa pharmacokinetic profile (opicapone regimen #2).

This suggests that more frequent levodopa daily dosing offers advantages in terms of motor fluctuations over less frequent dosing when opicapone is included as adjunct therapy.

Clinical Outcomes for LD/CD 500/125 mg QD5 without Opicapone Compared with LD/CD 400/100 mg QD4 Plus 50 mg Opicapone

### 12-Hour Patient ON/OFF Monitoring

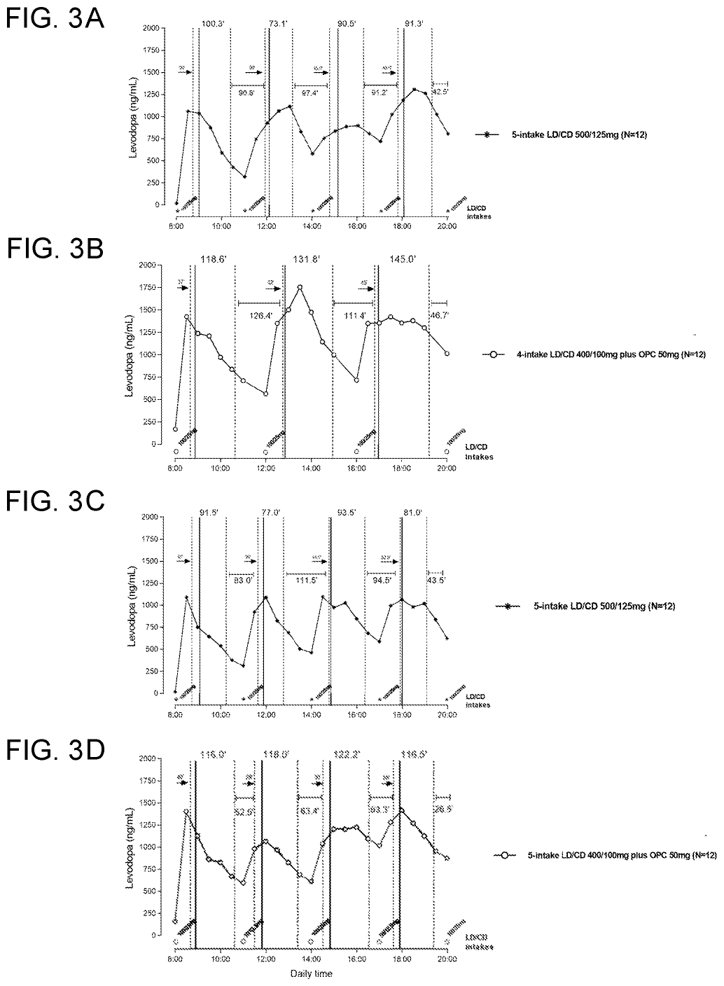

A 16%, non-significant, decrease in OFF-time and a 16% increase in ON-time were observed with 4-intake LD/CD 400/100 mg plus 50 mg opicapone compared with 5-intake LD/CD 500/125 mg without opicapone (see FIG. 3). Time to ON and time to best ON decreased by 12% and 18% respectively (decrease was significant for time to best ON, p=0.0439).

### 24-Hour Patient ON/OFF Monitoring

A 12%, significant, decrease in total OFF-time (p=0.0336) and a 11%, significant, increase in ON-time (p=0.0015) were observed with 4-intake LD/CD 400/100 mg plus 50 mg opicapone compared with 5-intake LD/CD 500/125 mg without opicapone (see FIG. 3). ON-time with troublesome dyskinesia decreased by ˜15% following the 4-intake LD/CD 400/100 mg plus 50 mg opicapone regimen.

### PGI-C

Approximately 70% of patients reported an improvement (very much/much/minimal improvement) on PGI-C with the 4-intake LD/CD 400/100 mg plus 50 mg opicapone regimen, with ˜33% of patients experiencing “very much/much improvement”.

See Table 3 for results.

Clinical Outcomes for LD/CD 500/125 mg QD5 without Opicapone Compared with LD/CD 400/100 mg QD5 Plus 50 mg Opicapone

### 12-Hour Patient ON/OFF Monitoring

A 45%, significant, decrease in OFF-time (p=0.0013) and a 47%, significant, increase in ON-time (p=0.0002) were observed with 5-intake LD/CD 400/100 mg plus 50 mg opicapone compared with 5-intake LD/CD 500/125 mg without opicapone (see FIG. 3). Time to ON and time to best ON decreased by 34% and 15% respectively (decrease was significant for time to best ON, p=0.0420).

### 24-Hour Patient ON/OFF Monitoring

A 24%, significant, decrease in total OFF-time (p=0.0056) and a 20%, significant, increase in ON-time (p=0.0007) were observed with 5-intake LD/CD 400/100 mg plus 50 mg opicapone compared with 5-intake LD/CD 500/125 mg without opicapone (see FIG. 3). ON-time with troublesome dyskinesia decreased by ˜36% following the 5-intake LD/CD 400/100 mg plus 50 mg opicapone regimen.

### PGI-C

Approximately 92% of patients reported an improvement (very much/much/minimal improvement) on PGI-C with the 5-intake LD/CD 400/100 mg plus 50 mg opicapone regimen, with ˜41.7% of patients experiencing “much improvement”.

See Table 3 for results.

TABLE 2
levodopa pharmacokinetic parameters following 2-week 5-intake (every 3 hours) daily oral administration
of LD/CD 500/125 mg without opicapone compared with 2-week 4-intake (every 4 hours) daily oral
administration of LD/CD 400/100 mg plus 50 mg opicapone or compared with 2-week 5-intake (every
3 hours) daily oral administration of LD/CD 400/100 mg plus 50 mg opicapone.

| | | | | | | | | | |
|---|---|---|---|---|---|---|---|---|---|
| Cₘₐₓ | C_{max, max} | tₘₐ | Cₘᵢₙ | C_{min, min} | AUC_{τ} | AUCₜₒₜₐₗ | t_{1/2} | FI | FI |
| (ng/mL) | (ng/mL) | (h) | (ng/mL) | (ng/mL) | (h*ng/mL) | (h*ng/mL) | (h) | (%) | (%) |
| n = 12 | n = 12 | n = 12 | n = 12 | n = 12 | n = 12 | n = 12 | n = 12 | n = 12 | n = 12 |

Arm A: 5-intake LD/CD 500/125 mg vs 4-intake LD/CD 400/100 mg plus OPC 50 mg
5-intake LD/CD 500/125 mg

| | | | | | | | | | | |
|---|---|---|---|---|---|---|---|---|---|---|
| 1^{st} LD/CD intake | 1393 | 2130 | 0.75 | 18.6 | 264.5 | 2076 | 10391 | 1.1⁺ | 202 | 166.9 |
| | (26.5) | (25.8) | (0.5-1.5) | (56.2) | (32.8) | (26.1) | (23.4) | (32.4) | (16.3) | (20.7) |
| 2^{nd} LD/CD intake | 1751 | | 1.5 | 266 | | 2637⁺⁺ | | 0.9⁺⁺ | 176⁺⁺ | |
| | (34.6) | | (0.5-2.5) | (31.2) | | (28.6) | | (27.1) | (34.2) | |
| 3^{rd} LD/CD intake | 1310 | | 1.5 | 403 | | 2850^{#} | | 1.3^{##} | 113^{#} | |
| | (36.6) | | (0.0-2.9) | (29.5) | | (29.3) | | (38.4) | (28.3) | |
| 4^{th} LD/CD intake | 1852 | | 1.5 | 559 | | 3414* | | 1.2* | 118* | |
| | (30.5) | | (0.5-2.5) | (35.7) | | (30.6) | | (7.6) | (19.5) | |

4-intake LD/CD 400/100 mg plus OPC 50 mg

| | | | | | | | | | | |
|---|---|---|---|---|---|---|---|---|---|---|
| 1^{st} LD/CD intake | 1814 | 2511 | 0.75 | 221 | 527.3 | 3705 | 13590 | 2.5 | 174 | 147.8 |
| | (33.0) | (34.4) | (0.5-2.0) | (67.9) | (41.1) | (33.1) | (32.8) | (20.3) | (20.9) | (13.0) |
| 2^{nd} LD/CD intake | 2235 | | 1.0 | 533 | | 4849 | | 2.1 | 144 | |
| | (30.8) | | (0.5-2.0) | (38.8) | | (31.9) | | (18.4) | (23.5) | |
| 3^{rd} LD/CD intake | 2007 | | 1.75 | 665 | | 5128* | | 2.7** | 110* | |
| | (48.5) | | (0.5-3.0) | (48.6) | | (33.3) | | (13.7) | (38.9) | |

Arm B: 5-intake LD/CD 500/125 mg vs 5-intake LD/CD 400/100 mg plus OPC 50 mg
5-intake LD/CD 500/125 mg

| | | | | | | | | | | |
|---|---|---|---|---|---|---|---|---|---|---|
| 1^{st} LD/CD intake | 1356 | 1999 | 0.5 | 18.1 | 267.0 | 1788^{$} | 9310 | 1.1⁺⁺ | 231^{$} | 175.5 |
| | (32.5) | (26.3) | (0.5-2.0) | (37.2) | (82.9) | (20.8) | (23.6) | (27.7) | (17.1) | (24.9) |
| 2^{nd} LD/CD intake | 1466 | | 1.0 | 283 | | 2269^{$} | | 1.4^{$} | 172^{$} | |
| | (32.7) | | (0.0-2.9) | (87.3) | | (39.6) | | (30.9) | (31.7) | |
| 3^{rd} LD/CD intake | 1530 | | 0.5 | 365 | | 2871* | | 1.5** | 148* | |
| | (46.0) | | (0.0-2.0) | (62.3) | | (37.7) | | (48.6) | (24.6) | |
| 4^{th} LD/CD intake | 1540 | | 1.5 | 455 | | 2871* | | 1.4* | 112* | |
| | (41.6) | | (0.5-3.0) | (41.1) | | (26.2) | | (48.9) | (39.6) | |

5-intake LD/CD 400/100 mg plus OPC 50 mg

| | | | | | | | | | | |
|---|---|---|---|---|---|---|---|---|---|---|
| 1^{st} LD/CD intake | 1664 | 1951 | 0.5 | 153 | 578.7 | 2657⁺ | 12044 | 2.1^{$} | 168⁺ | 103.7 |
| | (21.8) | (17.7) | (0.5-2.0) | (50.0) | (40.1) | (17.1) | (23.2) | (18.7) | (16.3) | (16.5) |
| 2^{nd} LD/CD intake | 1286 | | 1.0 | 579 | | 2397⁺ | | 2.8⁺ | 88.0⁺ | |
| | (27.1) | | (0.5-2.0) | (37.8) | | (21.3) | | (52.6) | (23.6) | |
| 3^{rd} LD/CD intake | 1572 | | 1.75 | 603 | | 4144^{#} | | 2.2^{##} | 91.6^{#} | |
| | (36.0) | | (0.5-2.9) | (35.8) | | (22.5) | | (28.1) | (28.0) | |
| 4^{th} LD/CD intake | 1557 | | 1.0 | 860 | | 3509^{$} | | 2.6^{$} | 60.2^{$} | |
| | (24.5) | | (0.5-2.0) | (25.9) | | (17.9) | | (36.1) | (27.4) | |

All values are expressed as mean (% CV) except for tₘₐₓ values that are expressed as median (range);
⁺11 subjects;
^{$}10 subjects
⁺⁺9 subjects;
*8 subjects;
**7 subjects;
^{#}6 subjects;
^{##}5 subjects.
AUC_{τ}, area under the concentration-time curve (AUC) over the dosing interval;
AUCₜₒₜₐₗ, AUC from zero hours to the last measurable time point;
Cₘₐₓ, maximum observed plasma concentration;
C_{max, max}, maximum Cₘₐₓ observed;
Cₘᵢₙ, minimum observed plasma concentration;
C_{min, min}, minimum Cₘᵢₙ observed (excluding first pre-dose);
FI, fluctuation index (calculated as: [(Cₘₐₓ − Cₘᵢₙ)/C_{avg}]*100);
CV, coefficient of variation;
LD/CD, levodopa/carbidopa;
OPC, opicapone;
tₘₐₓ, time taken to reach Cₘₐₓ;
SD, standard deviation t_{1/2}, terminal plasma half-life

TABLE 3
paired t-test analysis for main levodopa pharmacokinetic and efficacy parameters following
2-week 5-intake (every 3 hours) daily oral administration of LD/CD 500/125 mg without
opicapone compared with 2-week 4-intake (every 4 hours) daily oral administration
of LD/CD 400/100 mg plus 50 mg opicapone or compared with 2-week 5-intake (every 3
hours) daily oral administration of LD/CD 400/100 mg plus 50 mg opicapone.

| | | Geometric Mean (90% CI) | | | | |
|---|---|---|---|---|---|---|
| | | | Test | | | |
| | | Reference | (4/5-intake LD/ | Geometric Mean Ratio (GMR) | | |
| | | (5-intake LD/ | CD 400/100 mg + | | | p |
| Parameter | N | CD 500/125 mg) | OPC 50 mg) | Estimate | 90% CI | value |

Arm A: 5-intake LD/CD 500/125 mg vs 4-intake LD/CD 400/100 mg plus OPC 50 mg

| | | | | | | |
|---|---|---|---|---|---|---|
| AUCₜₒₜₐₗ (h · ng/mL) | 12 | 10126 (8940, | 12906 (10810, | 1.27 | 1.17, 1.38 | 0.0003 |
| | | 11469) | 15408) | | | |
| C_{max, max} (ng/mL) | 12 | 2059 (1782, | 2376 (1980, | 1.15 | 0.97, 1.37 | 0.3053 |
| | | 2380) | 2850) | | | |
| C_{min, min} (ng/mL) | 11 | 252.2 (211.4, | 487.1 (387.3, | 1.93 | 1.46, 2.55 | 0.0016 |
| | | 301.0) | 612.6) | | | |
| FI (%) | 12 | 163.7 (146.9, | 146.8 (137.6, | 0.90 | 0.80, 1.00 | 0.1095 |
| | | 182.3) | 156.6) | | | |
| 12-hour Time to ON (min) | 12 | 44.62 (38.75, | 39.48 (30.41, | 0.88 | 0.70, 1.12 | 0.3750 |
| | | 51.39) | 51.26) | | | |
| 12-hour Time to Best ON (min) | 12 | 57.62 (50.84, | 47.11 (37.79, | 0.82 | 0.70, 0.96 | 0.0439 |
| | | 65.31) | 58.73) | | | |
| 12-hour OFF-time (min) | 10 | 356.4 (313.2, | 299.3 (247.1, | 0.84 | 0.65, 1.1 | 0.2367 |
| | | 405.5) | 362.5) | | | |
| 12-hour ON-time (min) | 10 | 340.3 (292.0, | 394.3 (347.2, | 1.16 | 0.94, 1.4 | 0.2394 |
| | | 396.7) | 447.6) | | | |
| 24-hour total OFF-time (min) | 12 | 433.0 (386.4, | 382.1 (321.0, | 0.88 | 0.80, 0.97 | 0.0336 |
| | | 485.0) | 454.8) | | | |
| 24-hour total ON-time (min) | 12 | 476.5 (421.9, | 529.3 (473.8, | 1.11 | 1.06, 1.16 | 0.0015 |
| | | 538.2) | 591.3) | | | |

Arm B: 5-intake LD/CD 500/125 mg vs 5-intake LD/CD 400/100 mg plus OPC 50 mg

| | | | | | | |
|---|---|---|---|---|---|---|
| AUCₜₒₜₐₗ (h · ng/mL) | 12 | 9097 (8111, | 11745 (10386, | 1.29 | 1.23, 1.36 | <0.0001 |
| | | 10202) | 13282) | | | |
| C_{max, max} (ng/mL) | 12 | 1931 (1667, | 1920 (1741, | 0.99 | 0.90, 1.10 | 0.9259 |
| | | 2237) | 2119) | | | |
| C_{min, min} (ng/mL) | 11 | 218.9 (156.1, | 542.4 (442.7, | 2.48 | 1.90, 3.23 | <0.0001 |
| | | 307.0) | 664.5) | | | |
| FI (%) | 12 | 169.6 (145.9, | 102.3 (93.53, | 0.60 | 0.53, 0.69 | <0.0001 |
| | | 197,1) | 111.9) | | | |
| 12-hour Time to ON (min) | 10 | 41.84 (36.03, | 27.79 (18.64, | 0.66 | 0.48, 0.91 | 0.0420 |
| | | 48.59) | 41.44) | | | |
| 12-hour Time to Best ON (min) | 12 | 53.97 (47.84, | 45.99 (37.45, | 0.85 | 0.72, 1.01 | 0.1166 |
| | | 60.89) | 56.48) | | | |
| 12-hour OFF-time (min) | 9 | 376.9 (349.9, | 208.7 (164.2, | 0.55 | 0.44, 0.70 | 0.0013 |
| | | 406.0) | 265.3) | | | |
| 12-hour ON-time (min) | 9 | 333.1 (298.8, | 489.5 (441.1, | 1.47 | 1.32, 1.64 | 0.0002 |
| | | 371.2) | 543.1) | | | |
| 24-hour total OFF-time (min) | 12 | 420.3 (369.8, | 318.8 (261.8, | 0.76 | 0.66, 0.88 | 0.0056 |
| | | 477.6) | 388.3) | | | |
| 24-hour total ON-time (min) | 12 | 512.5 (470.9, | 614.6 (564.2, | 1.20 | 1.12, 1.29 | 0.0007 |
| | | 557.9) | 669.5) | | | |

Cₘₐₓ, maximum observed plasma concentration;
C_{max, max}, maximum Cₘₐₓ observed;
Cₘᵢₙ, minimum observed plasma concentration;
C_{min, min}, minimum Cₘᵢₙ observed (excluding first pre-dose);
AUCₜₒₜₐₗ, area under the curve (AUC) from zero hours to the last measurable time point;
FI, fluctuation index (calculated as: [(Cₘₐₓ − Cₘᵢₙ)/C_{avg}]*100);
CI, confidence interval;
LD/CD, levodopa/carbidopa;
OPC, opicapone

1. A method of treating Parkinson's disease in a patient who is receiving N doses of levodopa per day to provide a total daily dose of X mg of levodopa and who is starting to experience motor fluctuations or starting to show signs of “wearing-off”, the treatment comprising administering more than N doses of levodopa per day to provide a total daily dose of X mg of levodopa and administering a single daily dose of Y mg of opicapone, wherein X is from 100 to 1000, N is from 2 to 10 and Y is from 25 to 50.

2. (canceled)

3. (canceled)

4. The method according to claim 1, wherein the Parkinson's disease is idiopathic Parkinson's disease.

5. The method according to claim 1, wherein N is 3 to 6.

6. The method according to claim 5, wherein N is 4.

7. The method according to claim 1, wherein X is 300 to 600.

8. The method according to claim 7, wherein X is 400.

9. The method according to claim 1, wherein Y is 25 or 50.

10. The method according to claim 9, wherein the patient is receiving 4 doses of levodopa to provide a total daily dose of 400 mg per day and the treatment comprises administering 5 doses of levodopa per day to provide a total daily dose of 400 mg of levodopa and administering a single daily dose of 50 mg of opicapone.

11. The method according to claim 1, wherein the levodopa dosage amount is not the same for every dose.

12. The method according to claim 11, wherein the levodopa dosage amount is higher for odd numbered doses.

13. The method according to claim 11, wherein the levodopa dosage amount is higher for even numbered doses.

14. The method according to claim 10, wherein the 5 doses of levodopa to provide a total daily dose of 400 mg of levodopa are administered in the following dosing regimen: 100 mg, 50 mg, 100 mg, 50 mg and 100 mg.

15. The method according to claim 1, wherein the single daily dose of opicapone is administered at least 1 hour after the last dose of levodopa.

16. The method according to claim 1, wherein the patient is receiving N doses of a dopamine decarboxylase inhibitor (DDCI) to provide a total daily dose of Z mg of the DDCI, the method further comprising administering more than N doses of the DDCI per day to provide a total daily dose of Z mg of DDCI, wherein Z is from 50 to 250.

17. The method according to claim 16, wherein the DDCI is carbidopa or benserazide.

18. The method according to claim 16, wherein the DDCI doses are administered together with the levodopa doses.

19. The method according to claim 17, wherein the patient is receiving 4 doses of carbidopa to provide a total daily dose of 100 mg per day and the treatment comprises administering 5 doses of carbidopa per day to provide a total daily dose of 100 mg of carbidopa.

20. The method according to claim 1, wherein the treatment results in a reduction in “time to best ON”.

21. The method according to claim 1, wherein the treatment results in a decrease in ON-time with troublesome dyskinesia.

22. The method according to claim 1, wherein the treatment results in an improvement in the patient global impression of change.
